(19) United States
(12) Patent Application Publication
(10) Pub. No.: US 2013/0108503 A1
(43) Pub. Date: May 2, 2013

(54) Surgical Instrument Tray System and Method of Sterilization

(71) Applicants: Ryan Ramkhelawan, Lilburn, GA (US); Shawn Flynn, Norcross, GA (US)

(72) Inventors: Ryan Ramkhelawan, Lilburn, GA (US); Shawn Flynn, Norcross, GA (US)

(21) Appl. No.: 13/284,099

(22) Filed: Oct. 28, 2011

(51) Int. Cl. A61L 2/00; A61J 1/00

(52) U.S. Cl. 422/1; 211/85.13

(57) ABSTRACT

A surgical instrument support tray and method of sterilization comprising, in general, a first surface configured with a pattern of apertures, each aperture configured to support the surgical instrument, a second surface configured with a pattern of finger and positioned in parallel proximity to the first surface, wherein the second surface is configured to slide relative to the first surface and releasably lock the surgical instrument therebetween and, thus such apparatus, functions to removeably secure surgical instrument in group for identification, cleaning, sterilization and storage prior to surgery.

### TECHNICAL FIELD

The disclosure generally relates to surgical instrument receptacle and, more specifically, is related to a tray system for retaining a collection of surgical instruments for sterilization.

### BACKGROUND

Present day surgical procedures regularly use sets of pre-selected surgical instruments for a specified surgical procedure, such as clamps, hemostat, forceps, scissors, and retractors. These instruments are regularly grouped together to form a set. The set of surgical instruments is stored in a sterilized condition until required for surgery. Prior to sterilization the set of surgical instruments is subject to a time consuming multi-step sorting, identifying, grouping, cleaning and sterilization process. The set of surgical instruments is collected post operation, co-mingled in a wire mesh basket for transport to the central sterile processing area. Next, the wire mesh basket of surgical instruments is placed in a wash sink to brush and manually wash the surgical instruments to remove any foreign debris, such as tissue or dried body fluid. Next, the wire mesh basket of surgical instruments is transported to and run through an automated washer/decontaminator. Next, the wire mesh basket of surgical instruments is transported to and emptied out on a sorting table where a technician inspects, counts and sorts each surgical instrument into groupings of instruments for a specified surgical procedure. Next, pivoting or hinged scissor-like surgical instruments are commonly sequentially grouped using a stringer, bar or retaining rod positioned through both of the finger rings or ring handles, and the set is laid or positioned in a wire mesh basket. Next, the wire mesh basket of surgical instruments is placed in a sealed container and sealed before entering the sterilization machine. Next, the wrapped or containerized wire mesh basket of surgical instruments is placed in an industrial sterilization machine/autoclave for sterilization of the surgical instruments. Next, the sealed sterilization container of surgical instruments is stored until transported to an operating area for use as required. When needed the sterilized surgical instruments are transported to the operating room where the surgical instruments are removed from the wire mesh basket and arranged on a stand or instrument roll in a configuration that enables efficient transfer to a surgeon.

Moreover, instrument racks have been positioned in the wire mesh basket to maintain the instruments in a fixed side-by-side configuration ready for use in surgery and to permit contact with steam or sterilization fluids with all of the instruments surfaces which may not be exposed to sterilization if the instruments are stacked one on the other when laid in the wire mesh basket.

Much time is wasted during the cleaning, sorting, counting, and grouping procedure in the assembly and sterilization process. In addition, the onset of infectious diseases has dramatically increased the biohazard risk for patients, medical personnel and central sterile personnel and the demand for uncontaminated sterilized instruments. In addition, counting the surgical instruments before and after the surgical procedure is particularly important for ensuring that no instruments are left in the patient after the procedure has been completed.

Upon completion of surgery the surgical instruments are placed in a basket and sent to decontamination where decontamination staff hand wash and inspect the surgical instruments. Next the basket of surgical instruments is washed in a commercial washer/decontaminator and conveyed to an assembly area where an assembler inspects, sorts, and counts the surgical instruments. Sorted instruments are strung together with a stringer. After being strung instruments are placed in a basket then the basket is placed in a sterilization container, sealed then placed in the sterilization machine and sterilized in a commercial sterilizer using steam or other sterilant. The basket may be either wrapped or containerized. Once sterile the surgical instruments are placed in inventory for future use.

In addition, surgical instruments are often damaged when transported stacked one on the other as well as when the wire mesh basket of surgical instruments is emptied out on a sorting table post manual or automated washing or other pre-sterilization. This increases the likelihood of scratching or otherwise damaging the instruments, which are often delicate and expensive. Furthermore, such damage to the surgical instruments reduces the life expectancy of the surgical instruments resulting in increased medical costs to replace the surgical instruments.

Therefore, it is readily apparent that there is a recognizable unmet need for a surgical instrument tray system and method of sterilization that reduces the time spent cleaning, sorting, counting, identifying and grouping surgical instrument, extends the life expectancy of the surgical instruments, and decreases the contamination potential of the surgical instruments by maintaining the surgical instruments in a side-by-side open configuration during cleaning and sterilization.

### SUMMARY

Briefly described, in an example embodiment, the present apparatus and method overcomes the above-mentioned disadvantages and meets the recognized need for a surgical instrument support tray and method of sterilization comprising, in general, a first surface configured with a pattern of apertures, each aperture configured to support the surgical instrument, a second surface configured with a pattern of finger and positioned in parallel proximity to the first surface, wherein the second surface is configured to slide relative to the first surface and releasably lock the surgical instrument therebetween and, thus such apparatus, functions to removeably secure surgical instrument in group for identification, cleaning, sterilization and storage prior to surgery.

According to its major aspects and broadly stated, the present system meets the recognized need for a surgical instrument support tray configured to support a plurality of surgical instruments comprising a support member configured to support the plurality of surgical instruments, the support member further comprises a plurality of apertures formed in the support member and configured to receive the ring handles of the surgical instruments, and a retainer comb slideably positioned adjacent the support member, the retainer comb further comprises one or more finger members, wherein the finger members are positioned adjacent the plurality of apertures and configured to releasably retain the ring handles of the surgical instruments therein.

In a further exemplary embodiment of a method of efficient sterilization of surgical instruments having ring handles comprising the steps of positioning of the ring handles of the surgical instruments in a set of a plurality of apertures formed in a support member, wherein the surgical instruments are organized, retaining the ring handles of the surgical instruments with a retainer comb slideably positioned adjacent the support member, the retainer comb further comprises one or more finger members, wherein the finger members are positioned adjacent the plurality of apertures and configured to releasably retain the ring handles of the surgical instruments, and sterilizing the retained surgical instruments.

Accordingly, a feature of the surgical instrument support tray and method of sterilization is its ability to reduce the time required to identify, clean, sort, count, and group surgical instrument between surgeries.

Another feature of the surgical instrument support tray and method of sterilization is its ability to organize, protect and enable thorough cleaning and sterilization of surgical instruments.

Still another feature of the surgical instrument support tray and method of sterilization is its ability to accommodate a variety of surgical instruments, such as size, shape, angle of bend, tip type, instruments purpose and the like all in on support tray system.

Yet another feature of the surgical instrument support tray and method of sterilization is its ability to rapidly identify the appropriate instrument in the tray during sorting, counting, and grouping of the surgical instrument post surgery, during pre-wash, pressure washing, sorting, grouping, sterilization or storage for pre-surgical use.

Yet another feature of the surgical instrument support tray and method of sterilization is its ability to rapidly identify and select the appropriate surgical instrument during surgery without confusion and lapse of time and to also identify all surgical instrument post surgery.

Yet another feature of the surgical instrument support tray and method of sterilization is its ability to hold the surgical instruments in a desired position so as to make them easy to identify and select during surgery and post surgery.

Yet another feature of the surgical instrument support tray and method of sterilization is that individual surgical instruments are firmly held in a desired position preventing the instruments from coming into contact with one another or intermingled, therefore extending the life of each instrument.

Yet another feature of the surgical instrument support tray system and method of sterilization is selected sets of surgical instruments are firmly held in a desired position all in one tray assembly which may be positioned and stacked within a sterilization basket.

Yet another feature of the surgical instrument support tray system and method of sterilization is that selected surgical instruments may be accessed directly from the tray by a surgeon of surgery assistant.

Yet another feature of the surgical instrument support tray system and method of sterilization is its ability to reduce inefficiency and waste in turning around sterile surgical instruments as set forth in the Affordable Care Act.

Yet another feature of the surgical instrument support tray system and method of sterilization is its ability to decrease the loss of surgical instruments and decrease assembly time of surgical instrument sets.

Yet another feature of the surgical instrument support tray system and method of sterilization is its ability to reduce surgical instrument inventory or reduce the need for additional capital expenditures due to increased surgical volume as a result of more efficient processing time.

Yet another feature of the surgical instrument support tray system and method of sterilization is its ability to enable a uniform cleaning process reducing the potential for surgical site infection.

Yet another feature of the surgical instrument support tray system and method of sterilization is its ability to reduce the occurrences of malfunctions during the surgical procedure due to improperly inspected surgical instruments.

Yet another feature of the surgical instrument support tray system and method of sterilization is its ability to reduce the capital budget for repairs associated with care and handling of surgical instruments.

Yet another feature of the surgical instrument support tray system and method of sterilization is its ability to integrate the surgical instrument support tray as the tray top of box like container.

Yet another feature of the surgical instrument support tray system and method of sterilization is its ability to reduce the number of sharps being sent to central sterile and the reduction of accidents associated with needle/sharps injuries.

Yet another feature of the surgical instrument support tray system and method of sterilization is its ability to inventory surgical blades and needles after being disengaged therefrom.

Yet another feature of the surgical instrument support stray system and method of sterilization is its ability to color coordinate a group of trays within a surgical service for easy identification.

These and other features of the trim assembly will become more apparent to one skilled in the art from the following Brief Description of the Drawings, Detailed Drawings, Detailed Description and Claims when read in light of the accompanying drawings.

### BRIEF DESCRIPTION OF THE DRAWINGS

The present garment with carrying pouch and support system or the like will be better understood by reading the Detailed Description of the embodiments with reference to the accompanying drawings, in which like reference to numerals denote similar structures and refer to like elements throughout, and in which:

FIG. 1 is top views of an example surgical instrument for storage in the surgical instrument support tray system shown herein;

FIG. 2 is perspective views of an example embodiment box like tray bottom of the surgical instrument support tray system;

FIG. 3 is perspective views of an example embodiment box like tray top of the surgical instrument support tray system;

FIG. 4 is perspective views of an example embodiment stackable surgical instrument support tray system; and

FIG. 5 is a flow diagram of a method of sorting, identifying, grouping, counting, cleaning, pressure washing, sterilizing, and storing prior to surgical use.

It is to be noted that the drawings presented are intended solely for the purpose of illustration and that they are, therefore, neither desired nor intended to limit the disclosure to any or all of the exact details of construction shown, except insofar as they may be deemed essential to the claimed invention.

### DETAILED DESCRIPTION

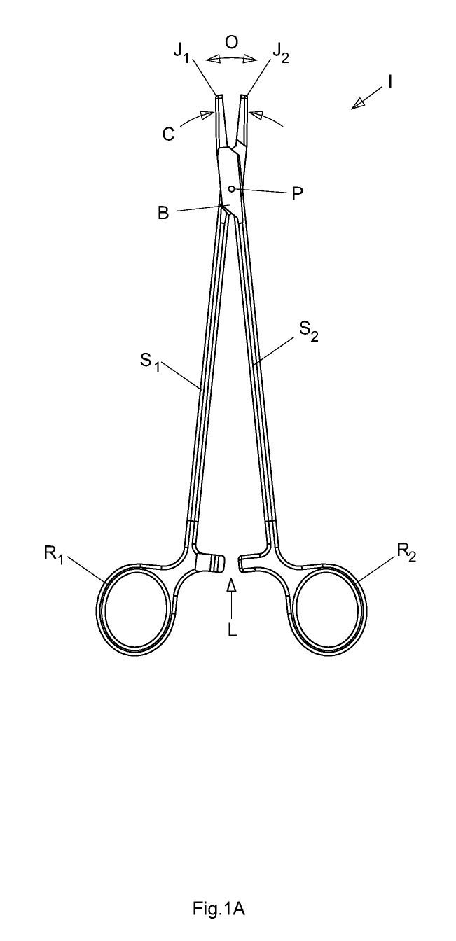

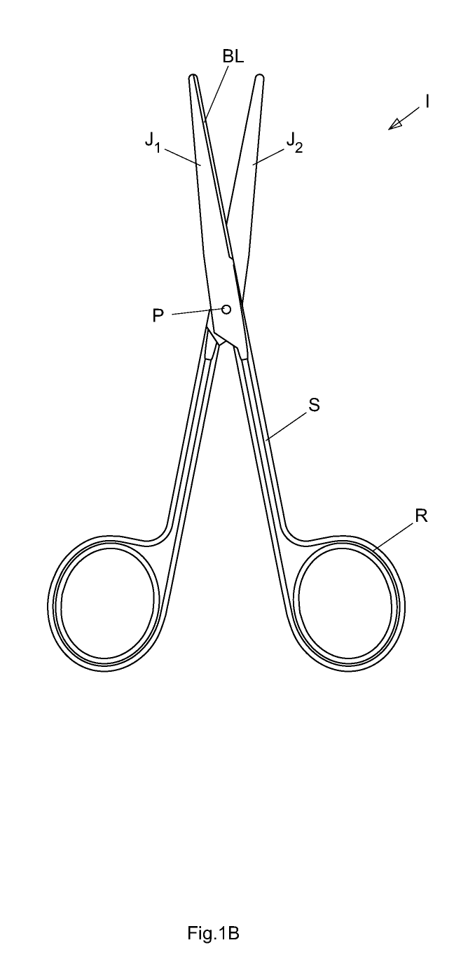

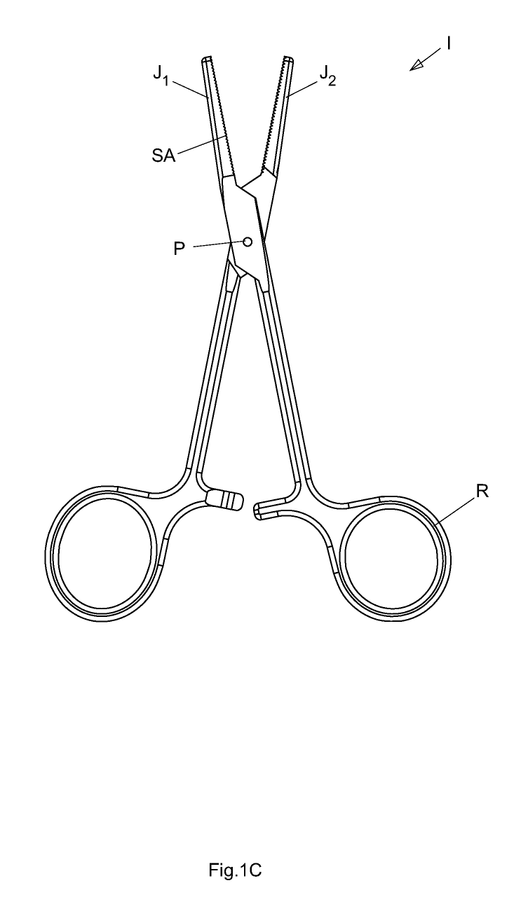

In describing the exemplary embodiments of the present disclosure, as illustrated in FIGS. 1-5 specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples.

Referring now to FIG. 1 there is illustrated a perspective view of an exemplary instrument such as surgical instrument I. Surgical instrument I preferably includes one or more finger loops or rings such as ring handles R, one or more levers, extension or support members S for positioning or extending surgical instrument I, and one or more moveable members M. Surgical instrument I are preferably formed of a suitable surgical material, such as stainless steel, aluminum, metal, metal alloys, shape memory alloys, carbon fibers, ceramic or the like, capable of providing structure to surgical instrument I. Preferably, the material includes other suitable characteristics, such as durability, rigidity, stain-resistance, bacteria-resistant, light weight, chemical inertness, oxidation resistance, ease of workability, or other beneficial characteristic understood by one skilled in the art.

Referring now to FIG. 1A an exemplary hemostat having moveable members M, which preferably includes a pair of opposing first jaw member J1 and second jaw members J2, jaw members, where at least one of the jaw members is movable relative to the other. Such jaw members may be pivotable about pivot point P, within box lock B, between first open position O in which the jaw members are disposed in a spaced relation relative to one another, and second closed position C in which the jaw members are configured to grasp or clamp something therebetween, such as tissue, or the like. Moreover, opposing first jaw member J1 and second jaw members J2 may be locked or held in position by a lock such as ratchet lock L preferably positioned on an extension stem or support member such as shank S1 and shank S2 between the two ring handles R. In use, for example a user positions their thumb in first ring handle R1 and their index or middle finger within second ring handle R2. By a user opening and closing their thumb and index or middle finger this causes first ring handle R1 and second ring handle R2 to move apart (first open position O) and together (second closed position C), accordingly. The movements of first ring handle R1 and second ring handle R2 causes shank S1 and shank S2 to pivot about pivot point P which results in first jaw member J1 and second jaw members J2 to likewise pivot about pivot point P between first open position O and second closed position C.

Referring now to FIG. 1B exemplary scissors, such as Mayo Surgical Scissors having moveable members M, which preferably includes a pair of opposing first jaw member J1 and second jaw members J2, jaw members, having sharpened blades BL are configured to cut something therebetween, such as tissue, or the like.

Referring now to FIG. 1C exemplary needle drive having moveable members M, which preferably includes a pair of opposing first jaw member J1 and second jaw members J2, jaw members, having extended jaws with increased gripping surface area SA which are configured to grasp or clamp something therebetween, such as a needle, or the like.

Further, surgical instruments I may include hemostats, forceps, clamps, scalpels, scissors, picks, retractors, hooks, clips, pliers, punches, curettes, specula and the like, which are generally of high precision and intricate construction and come in a variety of types, shapes and sizes, all of which may be used during a particular surgical procedure. A variety of surgical tools exists for each category of instruments. For example, considering only forceps, they come straight, left curved, right curved, serrated, cupped, etc. In addition, a range of medical, dental and veterinary tools have been developed for each discipline, such as in medicine sub-categories of surgical procedures include anesthesia, cardio, dermatology, ear nose & throat, hand (specific limbs), facial, ob/gyn, orthopedic and the like each discipline having a variety of specialty and common surgical instruments. Over the years, surgical procedures along with the type and quantity of surgical instruments used in a given procedure have become predominantly standardized. Many of the above-mentioned instruments are scissor action instruments, having a pair of ring handles R that are connected through a pivot P to working moveable members M. Preferably, lever, extension or support members such as shank S typically include ring handles R at the ends thereof to facilitate the opening and closing of moveable members M, such as first jaw member J1 and second jaw members J2 by a user's fingers.

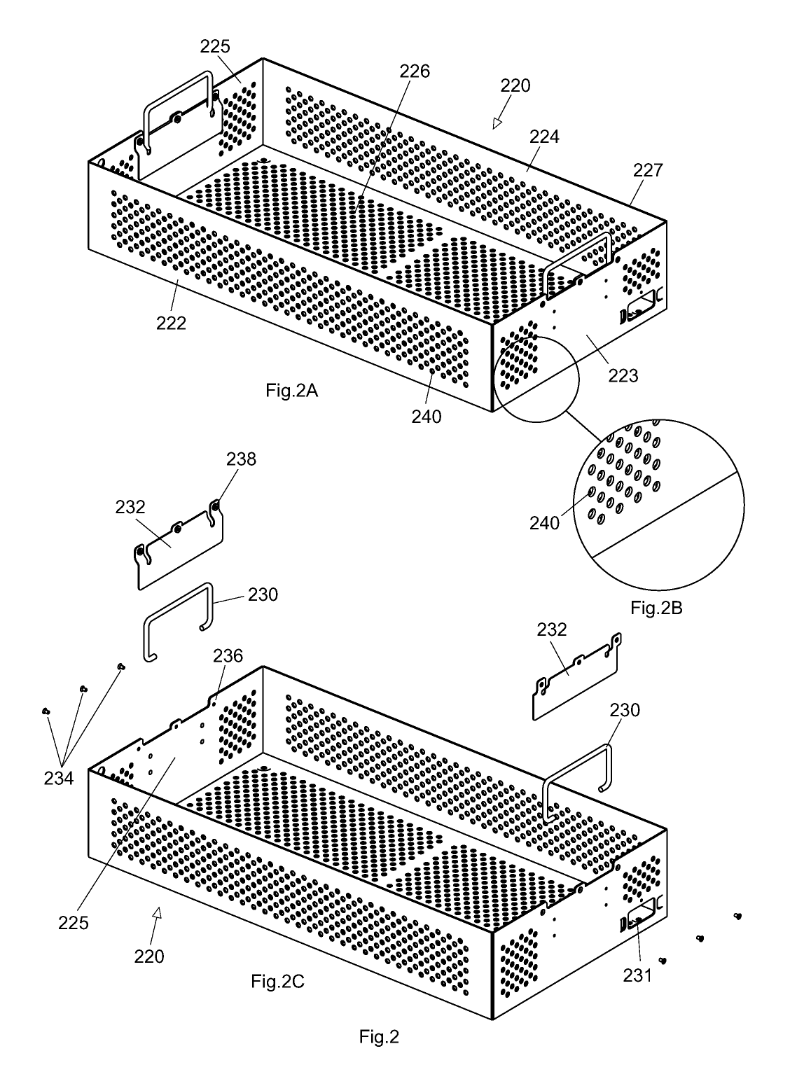

Referring now to FIG. 2A there is illustrated a perspective view of an example embodiment box like container such as tray bottom 220 of the surgical instrument support tray system 10. Preferably, tray bottom 220 may be configured as a basket-like housing or enclosure in which surgical instruments I may be positioned, sterilized, transported and stored for later use. Tray bottom 220 comprises four generally perpendicular, upwardly projecting, continuous planar member(s), surface(s) or side(s) such as such as side walls 222, 223, 224, 225 and bottom 226 arranged preferably as a rectangle or square and having an open top box. Side walls 222-225 preferably define a generally open top having an upper perimeter 227.

Side walls 222, 223, 224, 225 and bottom 226 are preferably formed of a suitable material, stainless steel, aluminum, metal, metal alloys, shape memory alloys, carbon fibers, ceramic or the like, capable of providing structure to sides walls 222, 223, 224, 225 and bottom 226. Preferably, the material includes other suitable characteristics, such as durability, rigidity, stain-resistance, bacteria-resistant, light weight, chemical inertness, oxidation resistance, ease of workability, or other beneficial characteristic understood by one skilled in the art.

It is contemplated herein that one or more sides walls 222, 223, 224, 225 and bottom 226 may be formed from a wire grid construction.

It is further contemplated herein that tray bottom 220 may include a variety of shapes and sizes to accommodate a variety of surgical instruments I.

Referring now to FIG. 2B there is illustrated any of side walls 222, 223, 224, 225 and bottom 226. Preferably side walls 222, 223, 224, 225 and bottom 226 may be formed or configured with a plurality or set of holes such as apertures 240 to enable steam or other sterilant to flow through apertures 240 formed in side walls 222, 223, 224, 225 and bottom 226 and permeate the entire interior of tray bottom 220. Furthermore, such sterilant may pass up, under and over the surgical instruments I positioned in tray bottom 220, thus ensuring effective sterilization.

Referring now to FIG. 2C there is illustrated an exploded component perspective view of an example embodiment box like tray bottom 220 of the surgical instrument support tray system. Preferably, tray bottom 220 includes two or more wire handles such as handle wire 230 configured generally in a u-shape. Handle wire 230 is preferably positioned approximate an interior side of one of side walls 222, 223, 224, or 225 (preferably 223 and 225) and removably affixed thereto and configured to enable swivel movement of handle wire 230 about a plate such as handle retainer plate 232. At least two handle retainer plates 232 are preferably affixed to side walls 223 and 225 by an attachment device such as machined screw 234 positioned through apertures 238 formed in handle retainer plates 232 and threaded into machined apertures 236 configured in side walls 223 and 225. Handle wires 230 are preferably affixed to side walls 223 and 225 to facilitate the lifting, carrying and positioning of tray bottom 220.

Furthermore, any of side walls 222, 223, 224, 225 and bottom 226 may include an identifying legend such as indicia 231 to enable identification of the surgical instruments I contained therein or the surgical procedure identified with the set or group of surgical instruments I contained within tray bottom 220, surgical kit identification, dates of sterilization, dates of expiration, model number, serial number, ownership and the like.

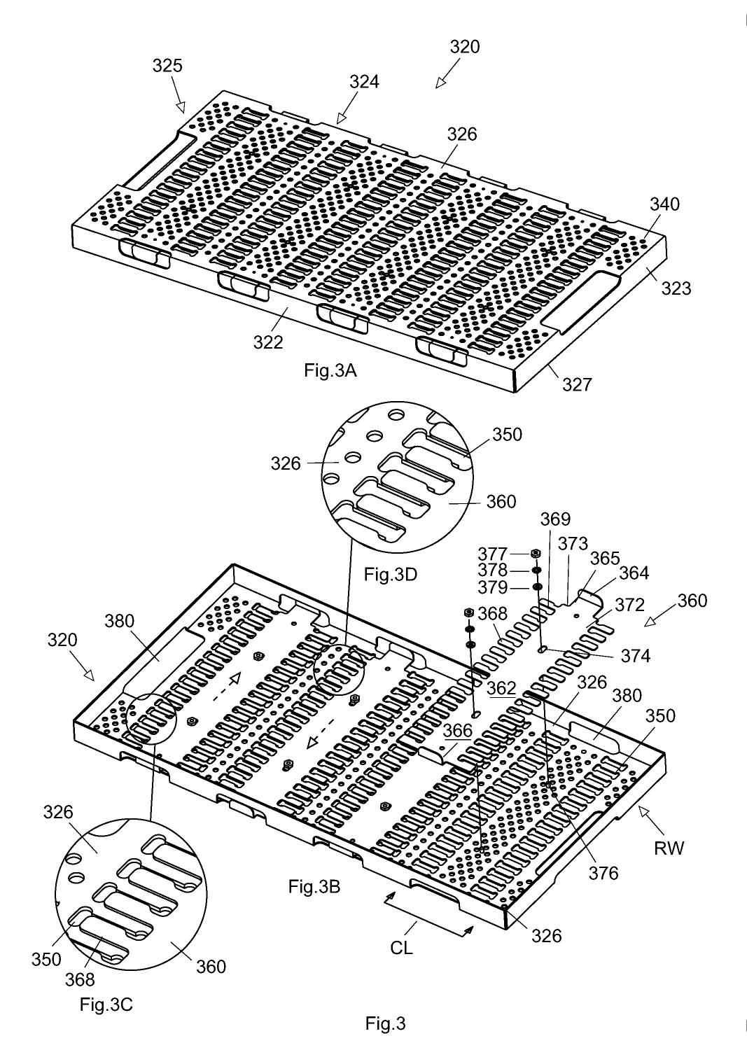

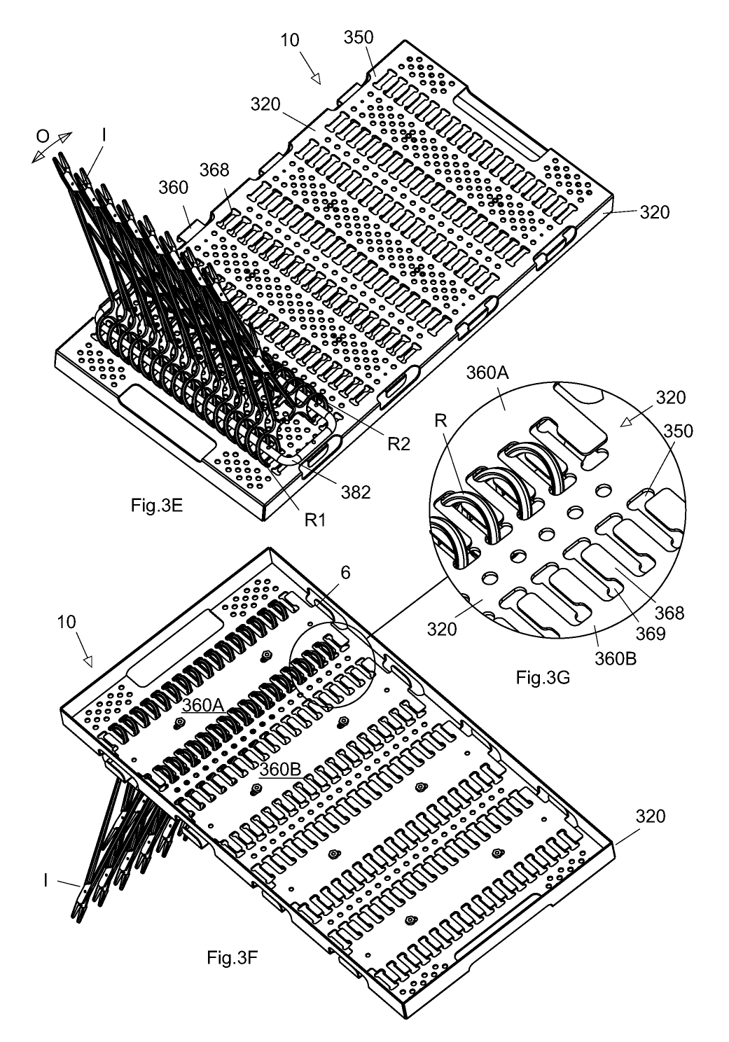

Referring now to FIG. 3A there is illustrated a perspective view of an example embodiment lid like tray top 320 of the surgical instrument support tray system 10. Preferably, tray top 320 may be configured as a box top or lid for tray bottom 220 in which surgical instruments I may be removably affixed in position, sterilized, transported and stored for later use. Tray top 320 comprises four generally perpendicular, downwardly projecting, continuous, planar member(s), surface(s) or side(s) arranged preferably as a rectangle or square. Side walls 322-325 preferably define a top having perimeter 227 configured to engage side walls 222-225 of tray bottom 220 to form an enclosure in which surgical instruments I removably affixed in position, sterilized, transported and stored for later use. Furthermore, any of side walls 322, 323, 324, 325 and/or top 326 may be configured as a support member such as top 326.

Likewise, side walls 322, 323, 324, 325 and top 326 may be formed or configured with a plurality or set of holes such as apertures 340 to enable steam or other sterilant to flow through apertures 340 formed in side walls 322, 323, 324, 325 and top 326 and permeate the entire interior of tray top 320 and into the interior of tray bottom 220. Furthermore, such sterilant may pass down through tray top 320 and over the surgical instruments I positioned in tray bottom 220, thus ensuring effective sterilization.

It is contemplated herein that tray bottom 220 and tray top 320 may be configured in shapes other than square and rectangle provided such configuration enables sterilization, transport and storage for later use of surgical instruments I.

Referring now to FIG. 3B there is illustrated a perspective exploded view of a 180 rotated tray top 320 of the surgical instrument support tray system 10. Preferably top 326 of tray top 320 includes regularly spaced rows and columns of a plurality or set of apertures such as slots 350 formed therethrough top 326. Slots 350, a pair making up a row, are preferably configured in a bone shape into which portions of surgical instrument I, preferably ring handles R, may be inserted and held in a desired and evenly spaced position preventing surgical instrument I from coming into contact with one another, intermingling, and enabling easy counting and sorting of surgical instruments I. Slots 350 are preferably formed in sets of two to a row to form one surgical instrument I positioning unit of surgical instrument support tray system 10. Moreover, a plurality of rows RW of pairs of slots 350 preferably form two columns CL to form a group or set of surgical instruments I positioning units of surgical instrument support tray system 10.

Furthermore, it is recognized herein that slots 350 may be configured in other shapes and sizes such as oval, ellipse or the like to accommodate a variety of sizes and shapes of portions of surgical instrument I.

Referring again to FIG. 3B there is illustrated one or more slideable latch device such as retainer comb 360. Preferably retainer comb 360, which generally includes a planar rectangular area such as base sheet 362 having a control lever such as tab 364 positioned on one or more opposing short ends of base sheet 362 such as first end 365 and second end 366 and is generally perpendicular and projecting upwardly from sheet 362. Tab 364 may be used to slide or position retainer comb 360 relative to tray top 320. It is recognized herein that tab 364 may be any size or configuration that enables a user to engage tab 364 and move or slide retainer comb 360 in a parallel plane relative to the interior surface of top 326 of tray top 320. Retainer comb 360 preferably includes one or more apertures such as oval slot 374 to slideably affix retainer comb 360 to top 326 of tray top 320 by passing therethrough slot 374 an attachment mechanism such as bolt 376 positioned through top 326 and oval slot 374, spacer 379, washer 378, and nut 377.

It is contemplated herein that other attachment mechanisms may be utilized to slideably affix retainer comb 360 to top 326.

Again side walls 322, 323, 324, 325 and top 326 of tray top 320 may be formed or configured with a plurality of apertures such as access slots 380 positioned around the perimeter between side walls 322, 323, 324, 325 and top 326. In use, access slots 380 are preferably configured to enable access to operate tab 364 and/or to enable lifting and/or positioning of tray top 320.

Furthermore, retainer comb 360 includes a plurality of fingers or finger members such as tooth 368 positioned along one or more long ends of base sheet 362 such as fist edge 372 and second edge 373 of retainer comb 360. Tooth 368 may include one or more indentations such as notch 369 positioned approximate the valley between two such teeth 368. Preferably, notch 369 is configured to surround ring handles R and secure in place ring handles R of surgical instrument I when retainer comb 360 is in a closed position.

Referring now to FIG. 3D there is illustrated an enlarged view of retainer comb 360 in the open position wherein surgical instruments I may be inserted into slots 350. Alternately, referring now to FIG. 3C there is illustrated an enlarged view of retainer comb 360 in the closed position wherein surgical instruments I may be mechanically/releasably retained in slots 350 by tooth 368 of retainer comb 360. In use, operation of tab 364 through access slots 380 (shown in FIG. 3B) enables retainer comb 360 to move between open and closed positions and collectively function to mechanically grip and release surgical instruments I positioned slots 350 within surgical instrument support tray system 10.

Side walls 322, 323, 324, 325, bottom 326 and retainer comb 360 are preferably formed of a suitable material, stainless steel, aluminum, metal, metal alloys, shape memory alloys, carbon fibers, ceramic or the like, capable of providing structure to sides walls 322, 323, 324, 325, bottom 326 and retainer comb 360. Preferably, the material includes other suitable characteristics, such as durability, rigidity, stain-resistance, bacteria-resistant, light weight, chemical inertness, oxidation resistance, ease of workability, or other beneficial characteristic understood by one skilled in the art.

Referring now to FIG. 3E there is illustrated a perspective view of the example embodiment shown in FIG. 3A lid like tray top 320 of the surgical instrument support tray system 10 shown with surgical instruments I retained in an organized, parallel relationship. In a preferred use ring handles R of surgical instrument I may be inserted into slots 350 and surgical instrument I may be mechanically/releasably retained in an organized, parallel relationship in slots 350 by tooth 368 of retainer comb 360 when retainer comb 360 is in the closed position to form a dual slot 350 locking mechanism, with tooth 368 of retainer comb 360, for each surgical instrument I held therein. Slots 350, a pair making up a row, may be spaced a desired distance apart to enable cleaning and sterilization while surgical instruments I are held in their open O position, and thus serves to aid in efficient and thorough cleaning and sterilization of the organized open position surgical instrument I. Further, another advantage of slots 350, a pair making up a row is surgical instrument support tray system 10 preferably reduces the number of sharps being sent to sterile central since ring handles R of surgical instrument I must be opened O to insert into slots 350. For example, if a surgical instrument I was used in conjunction with a suture (needle and thread) the needle will fall out when surgical instrument I is opened O allowing surgical staff to properly dispose of the sharp needle. Moreover, FIG. 3E further illustrates a retainer ring such as stringer 382 configured to pass through sets of ring handles R of surgical instrument I loaded into tray top 320 to retain the column set of surgical instrument I as a set when disengaged and removed from tray top 320.

It is recognized that tray top 320 having slots 350, notched tooth 368, and retainer comb 360 mechanically holds surgical instruments I in an organized, spaced, parallel, and slanted relationship (organized), and thus reduces the time required to identify, clean, sort, count, and group surgical instruments I between surgeries.

It is still further recognized that tray top 320 having slots 350, notched tooth 368, and retainer comb 360 enables tray top 320 to securely and neatly retain a group, set or plurality of surgical instruments I within the tray top 320 of surgical instrument support tray system 10.

It is still further recognized that tray top 320 having slots 350, notched tooth 368, and retainer comb 360 may accommodate a variety of surgical instrument I, such as size, shape, angle of bend, tip type, instruments purpose and the like all in one surgical instrument support tray system 10.

It is still further recognized that tray top 320 preferably provides a flat area bottom 326 to accommodate or position non-ring handle surgical instruments or large ring handle surgical instruments.

It is still further recognized that tray top 320 preferably is configured as a lid for tray bottom 220.

Moreover, tray bottom 220 is preferably configured to hold surgical instruments I in a desired position preventing the instruments from coming into contact with one another or intermingled, therefore extending the life of each instrument.

Referring now to FIG. 3F there is illustrated a perspective view of the example embodiment shown in FIG. 3B lid like tray top 320 of the surgical instrument support tray system 10 shown with surgical instruments I retained in an organized, parallel relationship. Similar to FIG. 3E ring handles R of surgical instrument I may be inserted into slots 350 and surgical instrument I may be mechanically/releasably retained in an organized, parallel relationship in slots 350 by tooth 368 of retainer comb 360A when retainer comb 360A is in the closed position. In the next parallel set to retain surgical instrument I retainer comb 360B is shown in the open position ready to receive ring handles R of surgical instrument I.

Referring now to FIG. 3G there is illustrated a partial close-up perspective view of the example embodiment lid like tray top 320 of the surgical instrument support tray system 10 shown with surgical instruments I retained in an organized, parallel relationship. Similar to FIG. 3F ring handles R of surgical instrument I may be inserted into slots 350 and surgical instrument I may be mechanically/releasably retained in an organized, parallel relationship in slots 350 by notch 369 of tooth 368 of retainer comb 360A when retainer comb 360A is in the closed position.

Preferably tray bottom 220 and tray top 320 are especially designed to removeably fit together to form a housing or enclosure in which surgical instruments I may be sterilized, transported and stored for later use. Post sterilization, tray bottom 220 and tray top 320 may be placed within a box-like enclosure having a removable top, which top can be clamped in position with respect to the base to form a sterile enclosure and stored for later surgical use. Post surgery the surgical instruments I may be stored in tray bottom 220 or individual returned to slots 350, and mechanically gripped by notched tooth 368 of retainer comb 360 ready for post surgery counting, identification, pre-cleaning, sterilization and storage.

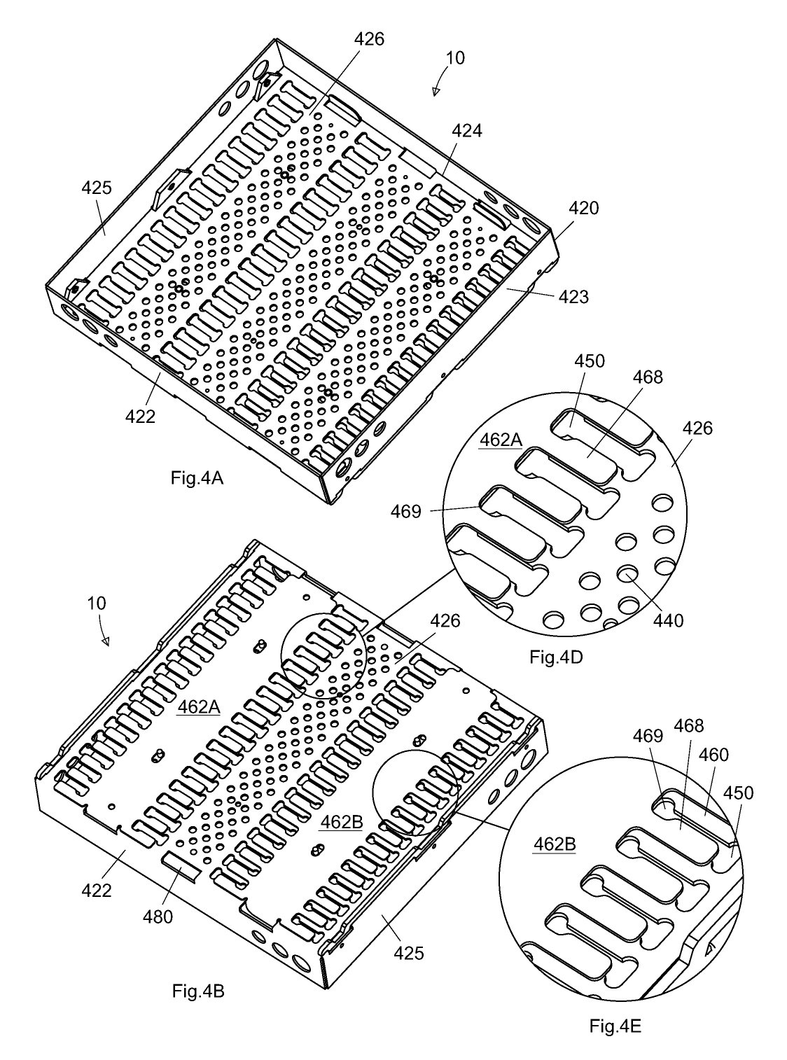

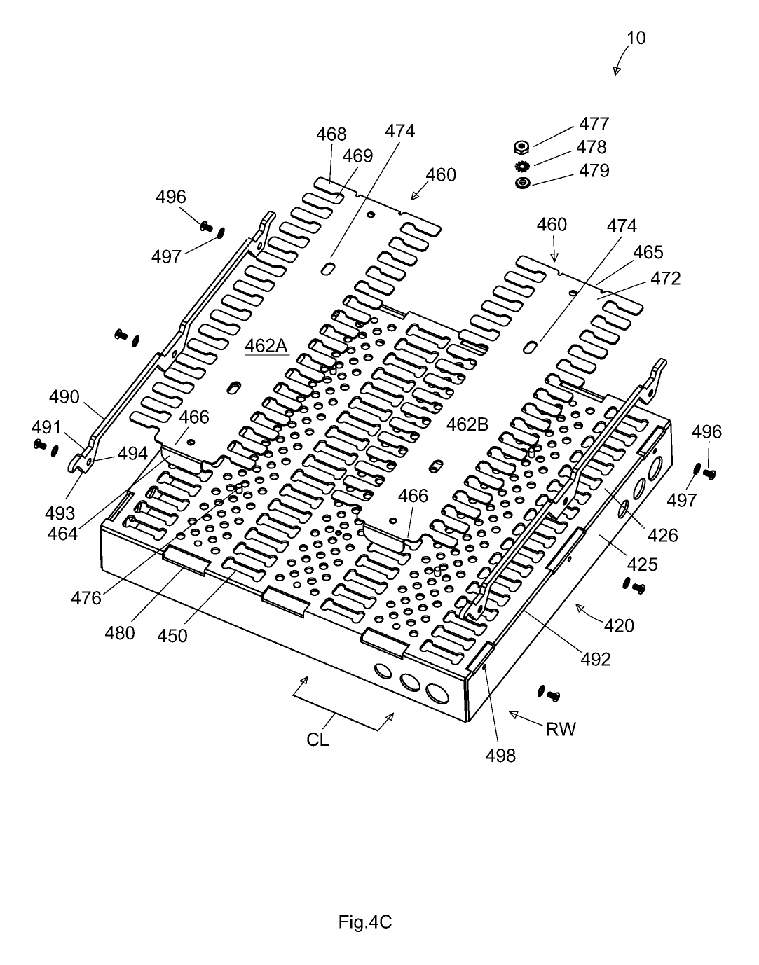

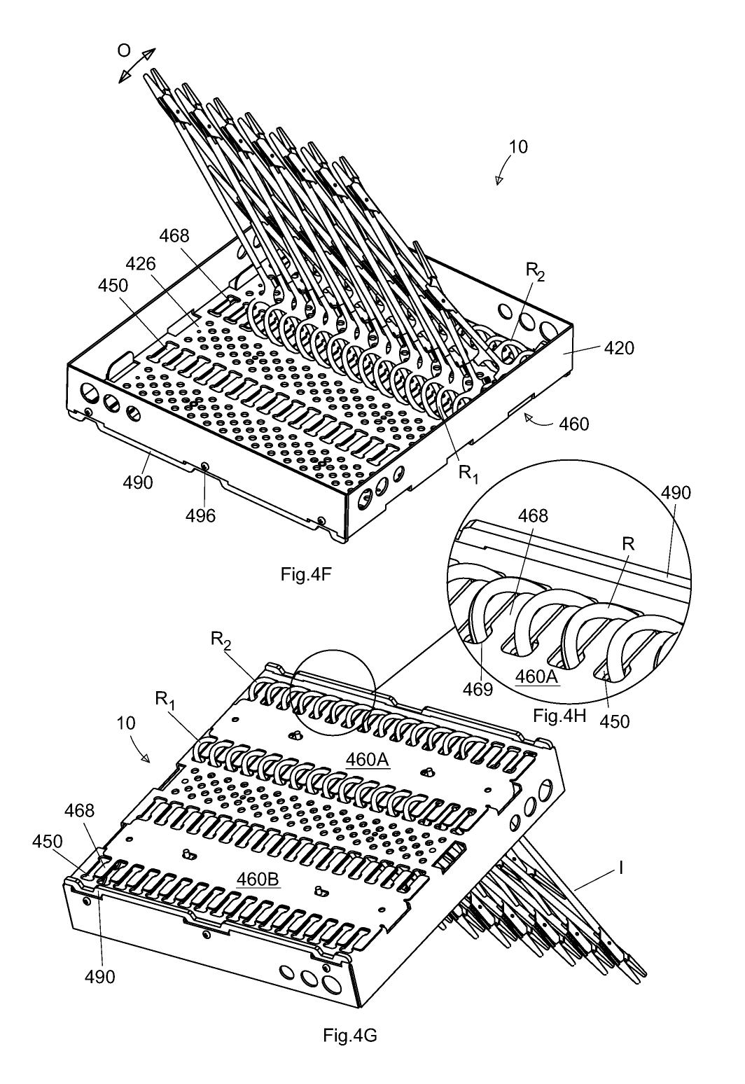

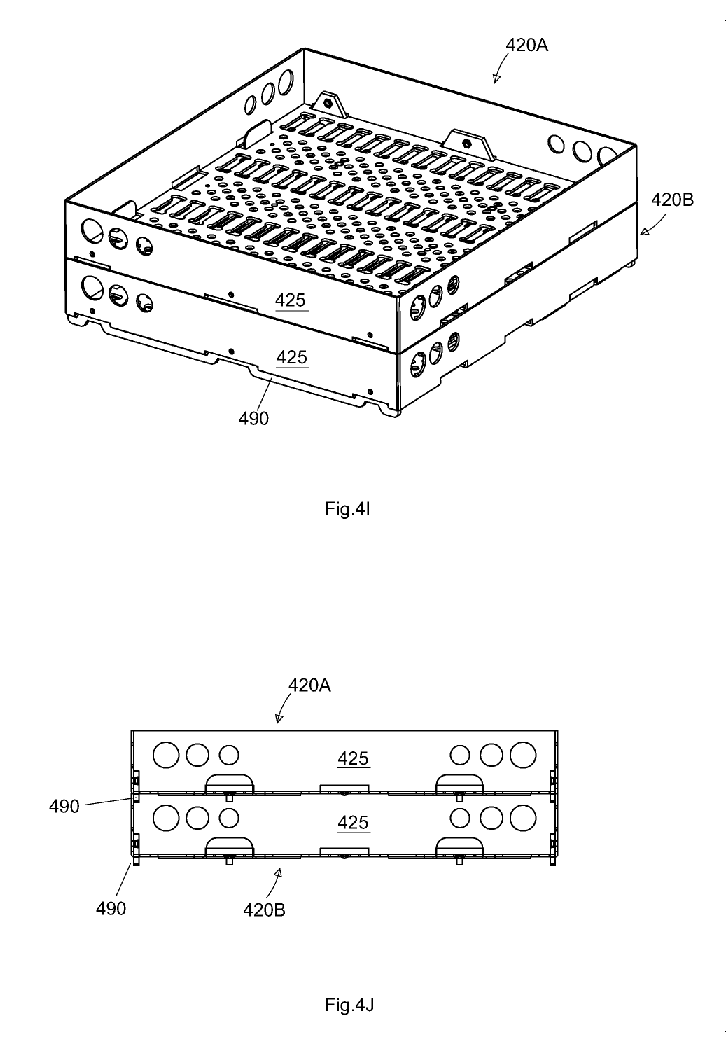

Referring now to FIG. 4A there is illustrated is perspective views of an example embodiment stackable tray basket 420 of surgical instrument support tray system 10. Preferably, a container such as tray basket 420 may be configured as a basket-like housing or enclosure in which surgical instruments I may be sterilized, transported and stored for later use. Tray basket 420 comprises four generally perpendicular, upwardly projecting, continuous planar member(s), surface(s) or side(s) such as side walls 422, 423, 424, 425 and bottom 426 arranged preferably as a rectangle or square as an open top box. Side walls 422-425 preferably define a generally open top and upper perimeter 427. Furthermore, any of side walls 422, 423, 424, 425 and/or bottom 426 may be configured as a support member such as bottom 426.

Side walls 422, 423, 424, 425 and bottom 426 are preferably formed of a suitable material, stainless steel, aluminum, metal, metal alloys, shape memory alloys, carbon fibers, ceramic 422, 423, 424, 425 and bottom 426. Preferably, the material includes other suitable characteristics, such as durability, rigidity, stain-resistance, bacteria-resistant, light weight, chemical inertness, oxidation resistance, ease of workability, or other beneficial characteristic understood by one skilled in the art.

It is contemplated herein that one or more sides walls 422, 423, 424, 425 and bottom 426 may be formed from a wire grid construction.

Referring now to FIG. 4B there is illustrated a perspective exploded view of a 180 rotated tray basket 420 of the surgical instrument support tray system 10. Preferably sides walls 422, 423, 424, 425 and bottom 426 may be formed or configured with a plurality or set of holes such as apertures 440 to enable steam or other sterilant to flow through apertures 440 formed in sides walls 422, 423, 424, 425 and bottom 426 and to permeate the entire interior of tray basket 420. Furthermore, such sterilant may pass up, under and over the surgical instruments I positioned in tray basket 420, thus ensuring effective sterilization.

It is contemplated herein that tray basket 420 may be configured in shapes and sizes other than square and rectangle provided such configuration enables sterilization, transport and storage for later use of surgical instruments I.

Referring now to FIG. 4C there is illustrated a perspective exploded view of a 180 rotated tray basket 420 of the surgical instrument support tray system 10. Preferably tray basket 420 includes regularly spaced rows and columns of a plurality or sets of apertures such as slots 450 formed therethrough tray basket 420. Slots 450, a pair making up a row, are preferably configured in a bone shape into which portions of surgical instrument I, preferably ring handles R (shown in FIG. 4F-H, may be inserted and held in a desired and evenly spaced position preventing surgical instrument I from coming into contact with one another, intermingling, and enabling easy counting and sorting of organized surgical instruments I. Slots 450 are preferably formed in sets of two to a row to form one surgical instrument I positioning unit of surgical instrument support tray system 10. Moreover, a plurality of rows RW of pairs of slots 35 preferably form two columns CL to form a group or set of surgical instruments I positioning units of surgical instrument support tray system 10.

Furthermore, it is recognized herein that slots 450 may be configured in other shapes and sizes such as oval, ellipse or the like to accommodate a variety of sizes and shapes of portions of surgical instrument I.

Referring again to FIG. 4C there is illustrated one or more slideable latch device such as retainer comb 460. Preferably retainer comb 460 generally includes a planar rectangular area such as base sheet 462 having a control lever such as tab 464 positioned on one or more opposing short ends of base sheet 462 such as first end 465 and second end 466 and is generally perpendicular and projecting downwardly. Tab 464 may be used to slide or position retainer comb 460 relative to tray basket 420. It is recognized herein that tab 464 may be any size or configuration that enables a user to engage tab 464 and move or slide retainer comb 460 in a parallel plane relative to the interior surface of bottom 426 of tray top 420. Retainer comb 460 preferably includes one or more apertures such as oval slot 474 to slideably affix retainer comb 460 to bottom 426 of side walls 422, 423, 424, 425 and bottom 426 by passing therethrough slot 474 an attachment mechanism such as bolt 476 positioned through bottom 426, spacer 479, washer 478, and nut 477.

It is contemplated herein that other attachment mechanisms may be utilized to slideably affix retainer comb 460 to top 426.

Again sides walls 422, 423, 424, 425 and bottom 426 of tray basket 420 may be formed or configured with a plurality of apertures such as access slots 480 positioned around the perimeter between side walls 422, 423, 424, 425 and bottom 426. In use, access slots 480 are preferably configured to enable access to operate tab 464 and/or to enable lifting and/or positioning of tray basket 420.

Furthermore, retainer comb 460 includes a plurality of fingers such as tooth 468 positioned along one or more long ends of base sheet 462 such as fist edge 472 and second edge 474. Tooth 468 may include one or more indentations such as notch 469 positioned approximate the valley between two such teeth 468. Preferably, notch 469 is configured to fit the configuration of and frictionally secure in place ring handles R of surgical instrument I.

Referring again to FIG. 4C there is illustrated a stacking bar or foot such as rail 490. Preferably rail 490 may be positioned approximate one or more transition edge 492 between side walls 422, 423, 424, 425 and bottom 426. For example, rail 490 may be positioned approximate transition edge 492 between side wall 425 and bottom 426. Moreover, rail 490 preferably includes one or more apertures such as hole 494 to affix rail 490 to side wall 425 by passing therethrough hole 494 an attachment mechanism such as bolt 496 positioned through washer 497 hole 494, and threaded into machined apertures such as hole 498 formed in side wall 425. Preferably, in use, rails 490 may be utilized to elevate one or separate stacked surgical instrument support tray system 10 to enable steam or other sterilant to flow under support tray system 10 and through apertures 440 formed in side walls 422, 423, 424, 425 and bottom 426 to permeate the entire interior of support tray system 10. Preferably rail 490 may be configured with one or more recesses 491 and one or more bumps 493 wherein recesses 491 and a bend such as bumps 493 from two such rails 490 are further configured to fit one in the other. Furthermore, sides walls 422, 423, 424, 425 and bottom 426 of tray basket 420 may be formed or configured with a plurality of apertures such as slots 499 positioned around the perimeter 492 between side walls 422, 423, 424, 425 and bottom 426. In use, slots 499 are preferably configured to accommodate bumps 493 to enable recess placement of rail 490 relative to perimeter 492 between side walls 422, 423, 424, 425 and bottom 426.

Referring now to FIG. 4D there is illustrated an enlarged view of retainer comb 460 in the open position wherein surgical instruments I may be inserted into slots 450. Alternately, referring now to FIG. 4E there is illustrated an enlarged view of retainer comb 460 in the closed position wherein surgical instruments I may be mechanically/releasably retained in slots 450 by tooth 468 of retainer comb 460. In use, operation of tab 464 through access slots 480 (shown in FIG. 4B) enables retainer comb 460 to move between open and closed positions and collectively function to mechanically grip and release surgical instruments I positioned within slots 450 of surgical instrument support tray system 10.

Side walls 422, 423, 424, 425 and bottom 426 and retainer comb 460 are preferably formed of a suitable material, stainless steel, aluminum, metal, metal alloys, shape memory alloys, carbon fibers, ceramic or the like, capable of providing structure to side walls 422, 423, 424, 425 and bottom 426. Preferably, the material includes other suitable characteristics, such as durability, rigidity, stain-resistance, bacteria-resistant, light weight, chemical inertness, oxidation resistance, ease of workability, or other beneficial characteristic understood by one skilled in the art.

Referring now to FIG. 4F there is illustrated a perspective view of the example embodiment shown in FIG. 4A tray basket 420 of the surgical instrument support tray system 10 shown with surgical instruments I retained in an organized, parallel relationship. In a preferred use ring handles R of surgical instrument I may be inserted into slots 450 and surgical instrument I may be mechanically/releasably retained in an organized, parallel relationship in slots 450 by tooth 468 of retainer comb 460 when retainer comb 460 is in the closed position to form a dual slot 450 locking mechanism, with tooth 468 of retainer comb 460, for each surgical instrument I held therein. Slots 450, a pair making up a row, may be spaced a desired distance apart to enable cleaning and sterilization while surgical instruments I are held in their open O position, and thus serves to aid in efficient and thorough cleaning and sterilization of organized surgical instruments I.

Further, another advantage of slots 450, a pair making up a row is surgical instrument support tray system 10 preferably reduces the number of sharps being sent to sterile central since ring handles R of surgical instrument I must be opened O to insert into slots 350. For example, if a surgical instrument I was used in conjunction with a suture (needle and thread) the needle will fall out when surgical instrument I is opened O allowing surgical staff to properly dispose of the sharp needle.

It is recognized that tray basket 420 having slots 450, notched tooth 468, and retainer comb 460 mechanically hold surgical instruments I in an organized, spaced, parallel, and slanted relationship, and thus reduce the time required to identify, clean, sort, count, and group surgical instruments I between surgeries.

It is still further recognized that tray basket 420 having slots 450, notched tooth 468, and retainer comb 460 enables tray basket 420 to securely and neatly retain a group, set or plurality of surgical instruments I within tray basket 420 of surgical instrument support tray system 10.

It is still further recognized that tray basket 420 having slots 450, notched tooth 468, and retainer comb 460 may accommodate a variety of surgical instrument I, such as size, shape, angle of bend, tip type, instruments purpose and the like all in one surgical instrument support tray system 10.

It is still further recognized that tray basket 420 preferably provides a flat area bottom 426 to position or accommodate non-ring handle surgical instruments or large ring handle surgical instruments.

Moreover, tray basket 420 is preferably configured to hold surgical instruments I in a desired position preventing the instruments from coming into contact with one another or intermingled (organized), therefore extending the life of each instrument.

Referring now to FIG. 4G there is illustrated a perspective view of a 180 rotated tray basket 420 of the surgical instrument support tray system 10 shown in FIG. 4F. Similar to FIG. 4F ring handles R of surgical instrument I may be inserted into slots 450 and surgical instrument I may be mechanically/releasably retained in an organized, parallel relationship in slots 450 by tooth 468 of retainer comb 460A when retainer comb 460A is in the closed position. In the next parallel set to retain surgical instrument I retainer comb 460B is shown in the open position ready to receive ring handles R of surgical instrument I.

Referring now to FIG. 4H there is illustrated a partial close-up perspective view of the example embodiment tray basket 420 of the surgical instrument support tray system 10 shown with surgical instruments I retained in an organized, parallel relationship. Similar to FIG. 4G ring handles R of surgical instrument I may be inserted into slots 450 and surgical instrument I may be mechanically/releasably retained in an organized, parallel relationship in slots 450 by notch 469 of tooth 468 of retainer comb 460A when retainer comb 460A is in the closed position.

Referring to FIG. 4I there is illustrated a perspective view of the example embodiment tray basket 420 of the surgical instrument support tray system 10 shown with one tray basket 420A stacked on another tray basket 420B. Preferably, rail 490 of tray basket 420B elevates tray basket 420B above any surface tray basket 420B may be placed upon or creates a separation between stacked trays to enable space between the surface and tray basket 420A for ring handles R of surgical instrument I and to enable steam or other sterilant to flow beneath bottom 426 and to permeate the entire ring handles R of surgical instrument I. In addition, FIG. 4J there is illustrated a side view of the example embodiment tray basket 420 of FIG. 4I of the surgical instrument support tray system 10 shown with one tray basket 420A stacked on another tray basket 420B.

In addition, rails 490 is preferably configured to be wide and smooth to enable surgical staff to utilize surgical instrument support tray system 10 on a mayo stand during surgery. Traditionally, surgical staff places a sterile cover and then sterile towels on a mayo stand which is positioned above the patient's legs for the purpose of placing surgical instruments I closer to the surgeon, i.e. a small work station during surgery. On the mayo stand surgical staff usually place a rolled sterile towel and place surgical instruments I on there side propped up by the towel to accomplish some organization. Preferably, surgical instrument support tray system 10 is configured to fit the surface area of the mayo stand so the surgical staff may utilize the organization and functionality of surgical instrument support tray system 10 during surgery. Moreover, rails 490 are configured with smooth surfaces and edges to prevent tears in the sterile drape used to cover the mayo stand.

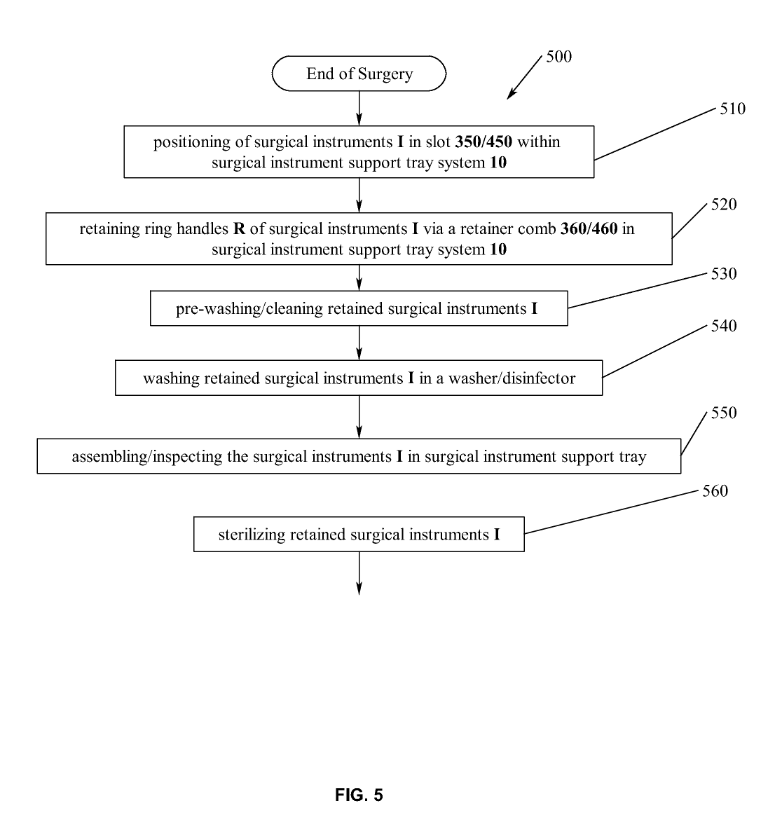

Referring to FIG. 5 there is illustrated a flow diagram 500 of a method of sorting, identifying, grouping, counting, cleaning, pressure washing, sterilizing, and storing prior to surgical use with decreased damage to surgical instruments, and decreased assembly time for sterile surgical instrument sets.

In block or step 510 and as described above in FIGS. 1-4, upon completion of surgery the operating personnel break down the surgical case and the surgical instruments I are sorted and ring handles R of surgical instrument I may be inserted into slots 350/450 and surgical instrument I may be identified, sorted and positioned in an organized, parallel relationship in slots 350/450 to form a set or group of surgical instruments I. Since surgical instruments I have been previously retained in parallel, even spaced with open jaw by surgical instrument support tray system 10 an assembler may quickly inspect, identify, sort, count, disengage any of the surgical instruments I for further inspection and operational testing, and grouping of surgical instruments I. Thus, surgical instrument support tray system 10 reduces the time spent cleaning, sorting, counting, identifying and grouping surgical instruments I, extends the life expectancy of the surgical instruments, and enables thorough cleaning of the surgical instruments.

In block or step 520, ring handles R of surgical instrument I may be mechanically retained by tooth 368/468 of retainer comb 360A/460A when retainer comb 360A/460A is engaged in the closed position. Herein, surgical instrument support tray system 10 firmly holds surgical instruments I in a desired position preventing surgical instruments I from coming into contact with one another or intermingled, therefore extending the life of each instrument. In addition, ring handles R of surgical instrument I may be strung together using stringer 382. Still further, when surgical instruments I are retained in parallel, evenly spaced and an open jaw position by surgical instrument support tray system 10 they may be sprayed and evenly coated with an enzymatic to begin breaking down post surgery contaminants.

In block or step 530, organized surgical instruments I may be pre-washed by submerging surgical instrument support tray system 10 with retained surgical instruments I into a detergent pre-wash solution or sonification system. In addition, surgical instruments I may be scrubbed or pressure washed to remove any visible contaminants remaining post surgery and inspected for completeness.

In block or step 540, surgical instrument support tray system 10 with retained parallel spaced and open jaw surgical instruments I may be washed in a commercial washer/disinfector. Since surgical instruments I are retained in parallel, evenly spaced and in an open jaw position this enables even access of detergents and uniform cleaning of surgical instruments I by a commercial washer/disinfector. Furthermore, the commercial washer/disinfector may convey surgical instrument support tray system 10 with retained surgical instruments I to an assembly area where an assembler inspects, identifies, sorts and counts surgical instruments I. Since surgical instruments I have been previously retained in parallel, even spaced with open jaw by surgical instrument support tray system 10 an assembler may quickly inspect, identify, sort, count, disengage any of the surgical instruments I for further inspection and operational testing, and finally grouping of surgical instruments I. Thus, surgical instrument support tray system 10 reduces the time spent cleaning, sorting, counting, identifying and grouping surgical instruments I, extends the life expectancy of the surgical instruments, and enables thorough cleaning and decontamination of the surgical instruments.

In block or step 550, surgical instruments I may be organized, counted, inspected and assembled while retained in surgical instrument support tray system 10. In addition, surgical instrument support tray system 10 preferably reduces the number of sharps being bundled or hidden with the surgical instruments I and being sent to sterile central.

In block or step 560, surgical instrument support tray(s) system 10 with retained parallel spaced and open jaw surgical instruments I may be as is, wrapped, containerized (placed in a sterilization container, such as a basket with detachable lid) or stacked for sterile processing/distribution. Surgical instrument support tray(s) system 10 with retained parallel spaced and open jaw surgical instruments I may be sterilized in a commercial sterilizer using steam or other sterilant. The sterilant may access surgical instruments I via a plurality or set of holes such as apertures 240/340/440 in surgical instrument support tray system 10 to enable thorough cleaning and sterilization of surgical instruments. Once sterilized, surgical instrument support tray(s) system 10 with retained parallel spaced and open jaw surgical instruments I may be placed in inventory for future surgical use.

Alternatively, after the assembler inspected and counted surgical instruments I in their locked position the assembler may unlock the strung surgical instruments I and place them in tray bottom 220 shown in FIG. 2 and affix tray top 320 to tray bottom 220 or position on embodiment stackable tray basket 420 of surgical instrument support tray system 10 per staff preference. Surgical instrument support tray system 10 with strung and unstrung surgical instruments I may be placed in a large sterilization container and sterilized in a commercial sterilizer using steam or other sterilant. The sterilant may access surgical instruments I via a plurality or set of holes such as apertures 240/340/440 in surgical instrument support tray system 10 to enable thorough cleaning and sterilization of surgical instruments. After sterilization is completed the sterilization container may be stored for future use.

In use, the operating room personnel set up for the surgical case. During surgery parallel spaced and open jaw surgical instruments I may be accessed directly from the disengaged instrument support tray system 10 by a surgeon of surgery assistant or arranged on a stand or instrument roll in a configuration that enables efficient transfer to a surgeon.

The foregoing description and drawings comprise illustrative embodiments of the present invention. Having thus described exemplary embodiments, it should be noted by those ordinarily skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one ordinarily skilled in the art to which this invention pertains having the benefit of the teachings presented in the foregoing descriptions and the associated drawings. Although specific terms may be employed herein, they are used in a generic and descriptive sense only and not for purposes of limitation. Moreover, the present invention having been described in detail, it should be understood that various changes, substitutions and alterations can be made thereto without departing from the spirit and scope of the invention as defined by the appended claims. Accordingly, the present invention is not limited to the specific embodiments illustrated herein, but is limited only by the following claims.

Therefore, at least the following is claimed:

1. A surgical instrument support tray configured to support a plurality of surgical instruments comprising:
a support member configured to support the plurality of surgical instruments, said support member further comprises a plurality of apertures formed in said support member and configured to receive the ring handles of the surgical instruments; and
a retainer comb slideably positioned adjacent said support member, said retainer comb further comprises one or more finger members, wherein said finger members are positioned adjacent said plurality of apertures and configured to releasably retain the ring handles of the surgical instruments therein.

2. The surgical instrument support tray of claim 1, wherein said plurality of apertures forms a pair of apertures configured to receive a pair of ring handles of the surgical instrument.

3. The surgical instrument support tray of claim 2, wherein said apertures of said pair of apertures are spaced apart, said pair of apertures configured to retain the surgical instruments in an open position.

4. The surgical instrument support tray of claim 2, further comprises two or more pairs of apertures, wherein said two or more pairs of apertures are spaced apart, said two or more pairs of apertures configured for maintaining an organized relationship between the surgical instruments.

5. The surgical instrument support tray of claim 1, wherein said support member is configured to have one or more perpendicular side walls to form a lid.

6. The surgical instrument support tray of claim 2, wherein said lid is configured as a top for a container of the surgical instruments.

7. The surgical instrument support tray of claim 6, wherein said lid and said container further comprise a plurality of holes configured to enable sterilant to flow therethrough.

8. The surgical instrument support tray of claim 1, wherein said support member and said retainer comb are positioned parallel to one another.

9. The surgical instrument support tray of claim 1, further comprises an attachment mechanism utilized to slideably attach said support member and said retainer comb together.

10. The surgical instrument support tray of claim 1, wherein said support member further comprises at least one access slot configured to allow access inside said support member.

11. The surgical instrument support tray of claim 10, wherein said retainer comb further comprises a control lever utilized to position said retainer comb relative to said support member.

12. The surgical instrument support tray of claim 11, wherein said control lever is accessible through said access slot.

13. The surgical instrument support tray of claim 1, wherein each of said plurality of apertures is configured as bone shaped.

14. The surgical instrument support tray of claim 1, wherein said retainer comb further comprises one or more finger members formed in said retainer comb, said finger members configured to releasably retain the ring handles of the surgical instruments in said apertures formed in said support member.

15. The surgical instrument support tray of claim 14, wherein said finger members are formed on an edge of said retainer comb.

16. The surgical instrument support tray of claim 14, wherein said finger members further comprise a notch formed in said finger members, said notch configured to surround the ring handles of the surgical instruments positioned in said apertures formed in said support member.

17. The surgical instrument support tray of claim 1, wherein said apertures formed in said support member are configured in regularly spaced rows and columns, said apertures utilized for maintaining an organized relationship between the surgical instruments.

18. The surgical instrument support tray of claim 1, wherein said support member is configured to have one or more perpendicular side walls to form a basket.

19. The surgical instrument support tray of claim 18, further comprises a rail positioned adjacent said support member, said rail configured to elevate said basket.

20. A method of efficient sterilization of surgical instruments having ring handles comprising the steps of:
positioning of the ring handles of the surgical instruments in a set of a plurality of apertures formed in a support member, wherein said surgical instruments are organized;
retaining the ring handles of the surgical instruments with a retainer comb slideably positioned adjacent said support member, said retainer comb further comprises one or more finger members, wherein said finger members are positioned adjacent said plurality of apertures and configured to releasably retain the ring handles of the surgical instruments; and
sterilizing the retained surgical instruments.

21. The method of claim 21, further comprises the step of pre-washing the retained surgical instruments.

22. The method of claim 21, further comprises the step of washing the retained surgical instruments.

23. The method of claim 21, further comprises the step of disinfecting the retained surgical instruments.

24. The method of claim 21, further comprises the step of assembling the surgical instruments in an organized manner.

25. The method of claim 21, reducing the time spent cleaning the surgical instruments.

26. The method of claim 21, reducing the time spent sorting the surgical instruments.

27. The method of claim 21, reducing the time spent counting the surgical instruments.

28. The method of claim 21, reducing the time spent identifying the surgical instruments.

29. The method of claim 21, reducing the time spent counting the surgical instruments.

30. The method of claim 21, reducing the time spent grouping the surgical instruments.

31. The method of claim 21, reducing the number of sharps bundled with the surgical instruments.

32. The method of claim 21, reducing the number of sharps related injuries.
